SEQUENCE LISTING (1 sequence)

SEQ ID NO: 1 - protein, 11 aa, Homo sapiens
  MISC_FEATURE (1)..(11): Synthetic
XPPGGSKVILF

(19) United States
(12) Patent Application Publication
(10) Pub. No.: US 2014/0154258 A1
(43) Pub. Date: Jun. 5, 2014

(54) PHARMACEUTICAL COMPOSITIONS CONTAINING ANTIBODIES TO NEUROPEPTIDE HEAD ACTIVATOR AND METHODS THEREOF

(71) Applicants: Igor Anatolievich Pomytkin, Moscow (RU); Valentin Antonovich Vinogradov, Moscow (RU); Anton Sergeevich Chernopyatko, Moscow (RU)

(72) Inventors: Igor Anatolievich Pomytkin, Moscow (RU); Valentin Antonovich Vinogradov, Moscow (RU); Anton Sergeevich Chernopyatko, Moscow (RU)

(21) Appl. No.: 13/577,660

(22) PCT Filed: Feb. 9, 2010
(86) PCT No.: PCT/RU10/00049

§ 371 Date: Aug. 8, 2012

(51) Int. Cl. C07K 16/18

(52) U.S. Cl. CPC C07K 16/18
USPC 424/139.1

(57) ABSTRACT

This invention relates to pharmaceutical compositions comprising antibodies that specifically bind to the endogenous neuropeptide head activator (NHA) having the sequence of pGlu-Pro-Pro-Gly-Gly-Ser-Lys-Val-Ile-Leu-Phe and neutralize the function of NHA thereby. The invention also provides methods for neutralizing NHA function, and particularly for treating NHA-related disorders (e.g., cancer or cardiovascular diseases) by administering to mammals of the pharmaceutical compositions.

### FIELD OF THE INVENTION

The invention relates to pharmaceutical compositions comprising antibodies that specifically bind to the neuropeptide head activator (NHA) and neutralize the function of NHA thereby. Methods for treating cancer with the compositions are also described.

### BACKGROUND OF THE INVENTION

An undecapeptide having an amino acid sequence of pGlu-Pro-Pro-Gly-Gly-Ser-Lys-Val-Ile-Leu-Phe, wherein pGlu denotes pyroglutamic acid, was originally isolated from the freshwater coelenterate hydra and subsequently found in animals, including humans. The undecapeptide plays a role in normal tissue morphogenesis in animals from coelenterates to humans. The undecapeptide is known in the art as hydra head activator or neuropeptide head activator. At cellular level, the undecapeptide acts as the potent mitogen in G2-mitosis transition and promotes proliferation of different types of cells. Schaller H C, Bodenmuller H. PNAS, 1981, 78(11): 7000-7004. Bodenmuller H, Schaller H C. Nature, 1981, 293:579-580. Schaller H C et al., EMBO J, 1989, 8(11):3311-3318.

Frequently, the endogenous neuropeptide head activator (NHA) is involved in the development of pathological conditions and disorders. Pathologically high levels of the undecapeptide were found in the blood of patients with tumors in peripheral locations, especially in tumors of gastrointestinal tract and/or of neuroendocrine origin. The strong correlation between tumorigenesis and elevated NHA levels was identified. The complete tumor removal resulted in decrease of NHA levels in blood to normal values, whereas incomplete tumor resection was accompanied by a marked decrease of the undecapeptide in blood. Schaller H C et al., J Neurooncol, 1988, 6:251-258. Winnikes M et al., Eur J Cancer 1992, 28(2-3): 421-4. Elevated NHA levels in blood were shown to induce undesirable proliferation of non-tumor cells, e.g. development of the myocardial hypertrophy. Fedoseev V A et al., Biull Eksp Biol Med 1993, 115(3):307-9; 1993, 116(9):316-8. Thus, there is a need to protect mammals, including humans being, from pathologically elevated levels of the endogenous neuropeptide head activator for the preventing or treating hyperproliferative disorders, including tumors.

U.S. Pat. No. 4,457,917 discloses the undecapeptide having amino acid sequence of pGlu-Pro-Pro-Gly-Gly-Ser-Lys-Val-Ile-Leu-Phe and pharmaceutical compositions comprising this undecapeptide for cell-growth stimulating action. However, no published or disclosed in the art related to the use of antibodies against the neuropeptide head activator for the treatment or prevention of a disease associated with pathologically elevated levels of an endogenous NHA.

Non-obviously from the art, we found that antibodies against the neuropeptide head activator are useful for treating cancer.

It is an object of the present invention to provide pharmaceutical compositions comprising an antibody that specifically binds a peptide of the formula pGlu-Pro-Pro-Gly-Gly-Ser-Lys-Val-Ile-Leu-Phe and methods thereof.

### DETAILED DESCRIPTION OF THE INVENTION

The present invention provides a pharmaceutical composition comprising an antibody that specifically binds a peptide of the formula pGlu-Pro-Pro-Gly-Gly-Ser-Lys-Val-Ile-Leu-Phe (SEQ ID NO: 1) and a pharmaceutically acceptable carrier.

As used herein, the term “antibody” refers to an intact monoclonal or polyclonal antibody or an antigen-binding portion thereof that competes with the intact antibody for specific binding. See generally, Fundamental Immunology, Ch. 7 (Paul, W., ed., 2nd ed. Raven Press, N.Y. (1989)) (incorporated by reference in its entirety for all purposes). Antigen-binding portions may be produced by recombinant DNA techniques or by enzymatic or chemical cleavage of intact antibodies. In some embodiments, antigen-binding portions include Fab, Fab', F(ab')₂, Fd, Fv, dAb, and complementarity determining region (CDR) fragments, single-chain antibodies (scFv), chimeric antibodies, diabodies and polypeptides that contain at least a portion of an antibody that is sufficient to confer specific antigen binding to the polypeptide.

In some preferred embodiments of the present invention, the antibody is an isolated antibody.

As used herein, the term “isolated antibody” refers to antibodies that have been identified and separated and/or recovered from a component of its natural environment. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present.

In some preferred embodiments of the present invention, the antibody is a monoclonal antibody.

As used herein, the term “monoclonal antibody” refers to identical antibodies directed against a single antigen determinant of an antigen, in this case the endogenous peptide having the sequence SEQ ID NO: 1. The monoclonal antibodies (mAbs) of the invention can be produced by a variety of techniques, including conventional monoclonal antibody methodology, e.g., the standard somatic cell hybridization technique of Kohler and Milstein (1975, Nature 256:495). Such monoclonal antibodies include, but are not limited to, mouse antibodies; engineered antibodies such as humanized, chimeric, or fully human antibodies produced by phage display technology or of transgenic mice; and derivatives thereof such as antibody fragments, immunoconjugates and Fc fusions. In practicing the invention, the monoclonal antibody to the peptide SEQ ID NO: 1 may be produced by recombinant DNA technology in a mammalian cell culture, e.g. Chinese Hamster Ovary cells.

In some preferred embodiments of the present invention, the content of the antibody in pharmaceutical compositions of the present invention is from 0.01 to 70 wt. %.

The compositions of the present invention can comprise optional ingredients. Such optional ingredients generally are used individually at levels from about 0.0005% to about 10.0%, preferably from about 0.005% to about 1.0% by weight of the composition. Examples of suitable optional ingredients include, but are not limited to, carriers, solvents, buffers, emulsifiers, stabilizers, and preservatives.

As used herein, the term “pharmaceutically acceptable carrier” refers to any ingredient having no therapeutic activity and being nontoxic and thus suitable as carrier. Nonexclusive suitable carriers will include any of the carriers commonly used in pharmaceutical products, such as, for example, water for injections, microcrystalline cellulose, lactose and starch.

Pharmaceutical compositions of the invention may be prepared by standard techniques well known to those skilled in the art. Such procedures include, but are not limited to, mixing the antibody to the peptide of the formula SEQ ID NO: 1 with other ingredients of the composition in conventional manner. Accordingly, the antibody can be formulated as the pharmaceutical composition using pharmaceutically-acceptable carriers, excipients, diluents, auxiliary agents or other ingredients routinely provided in pharmaceutical compositions by one of ordinary skill in the art and include formulations for immediate release and for sustained release, e.g., microencapsulation. The present pharmaceutical composition can be administered by any convenient route including intravenous, subcutaneous, intramuscular, oral, oromucosal, or other parenteral or internal route. Similarly the compositions can be administered as a single dose or divided into multiple doses for administration. Such schedules are readily determined by the one ordinary skilled in the art.

Further, the present invention provides a method for treating cancer in a mammal, the method comprising a step of administering to the animal an effective amount of the pharmaceutical composition of the present invention.

In practicing the methods of the present invention, an effective amount of the compositions of the present invention may be administered by a variety of routes including, but are not limiting to, injections, e.g. intravenous, subcutaneous, or intramuscular; topical application to the mucosal epithelia; intranasal, and oral administration. Effective amounts of the composition of present invention may vary on mammal species and route of administration and is expected to vary from about 0.01 mg/kg body weight per day to about 100 mg//kg per day. Preferred amounts may be determined by one skilled in the art.

As used herein, the term “mammal” refers to humans (male or female) and companion animals, e.g., dogs, cats and horses.

Since the endogenous peptide SEQ ID NO: 1 is ubiquitously distributed in mammals and represents itself the universal mitogen for different types of cells, the compositions of the invention may be used for the prevention or treatment of a broad spectrum of neoplasms (cancer).

Nonexclusive examples of neoplasms include adenoma, adenomatous polyposis coli, angiofibroma, arachnoid cysts, astrocytoma, basal cell nevus syndrome, bone neoplasms, bowen's disease, breast cyst, breast neoplasms (breast cancer), breast neoplasms, male, burkitt lymphoma, carcinoid tumor, carcinoma, carcinoma, basal cell, carcinoma, merkel cell, cementoma, chalazion, choledochal cyst, chondroma, chondrosarcoma, chordoma, craniopharyngioma, cysts, dentigerous cyst, dermoid cyst, digestive cystem neoplasms, ear neoplasms, endocrine gland neoplasms, endometrial neoplasms, ependymoma, epidermal cyst, Epstein-Barr virus Infections, eye neoplasms, fibromatosis, juvenile hyaline, gastrointestinal neoplasms, gastrointestinal stromal tumors, genital neoplasms, glioblastoma, glioma, hamartoma, hamartoma syndrome, multiple head and neck neoplasms, hemangioma, cavernous, hemangiosarcoma, histiocytoma, benign fibrous, histiocytoma, malignant fibrous, Hodgkin disease, Hutchinson's melanotic freckle, hydatidiform mole, insulinoma, intestinal neoplasms, Krukenberg tumor, Lambert-Eaton myasthenic syndrome, laryngeal neoplasms, leiomyoma, leiomyosarcoma, leukemia, lipoma, lung neoplasms, lymphangioleiomyomatosis, lymphangioma, lymphoma, Non-Hodgkin lymphoma, mediastinal cyst, medulloblastoma, melanoma, meningioma, mesothelioma, mouth neoplasms, multiple myeloma, myoma, myxoma, Nelson syndrome, neoplasm metastasis, nervous system neoplasms, neurilemmoma, neuroblastoma, neuroendocrine tumors, neurofibromatoses, neuroma, odontogenic tumors, osteosarcoma, otorhinolaryngologic neoplasms, ovarian cysts, ovarian neoplasms, Paget's disease, pancreatic neoplasms, papilloma, paraganglioma, paraneoplastic syndromes, Peutz-Jeghers syndrome, pheochromocytoma, pilonidal sinus, polycystic ovary syndrome, popliteal cyst, precancerous conditions, prostatic neoplasms (prostate cancer), proteus syndrome, pseudomyxoma peritonei, ranula, rectal neoplasms, respiratory tract neoplasms, retinoblastoma, rhabdoid tumor, rhabdomyosarcoma, sarcoma, skin neoplasms, Sturge-Weber syndrome, Tarlov cysts, teratoma, testicular neoplasms, thoracic neoplasms, thymoma, thyroid neoplasms, thyroid nodule, tonsillar neoplasms, trophoblastic neoplasms, tuberous sclerosis, tumor virus infections, urinary bladder neoplasms, urologic neoplasms, uterine cervical dysplasia, uterine cervical neoplasms, Waldenstrom macroglobulinemia, warts, wilms tumor, vulvar neoplasms, xerodenna pigmentosum, and Zollinger-Ellison syndrome.

Further, the present invention provides a method for treating a cardiovascular disease, the method comprising a step of administering to the mammal an effective amount of the pharmaceutical composition of the present invention.

As used herein, “cardiovascular disease” has the meaning commonly used in the field, and includes, but is not limited to, the following diseases or conditions: thromboembolic disorders, including arterial cardiovascular thromboembolic disorders, venous cardiovascular thromboembolic disorders, and thromboembolic disorders in the chambers of the heart; ahtherosclerosis; restensosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); first or recurrent myocardial infarction; acutemyocardial infarction (AMI); myocardial infarction; non-Q wave myocardial infarction; non-STE myocardial infarction; coronary artery disease; cardiac ischemia; ischemia; ischemic sudden death; transient ischemic attack; stroke; atherosclerosis; peripheral occlusive arterial disease; venous thrombosis; deep vein thrombosis; thrombophlebitis; arterial embolism; coronary arterial thrombosis; cerebral arterial thrombosis; cerebral embolism; kidney embolism; pulmonary embolism; thrombosis resultingfrom (a) prosthetic valves or other implants, (b) indwelling catheters, (c) stents, (d) cardiopulmonary bypass, (e) hemodialysis, or (f) other procedures in which blood is exposed to an artificial surface that promotes thrombosis; thrombosis resulting from atherosclerosis, surgery or surgical complications, prolonged immobilization, arterial fibrillation, congenital thrombophilia, cancer, diabetes, effects of medications or hormones, and complications of pregnancy; cardiac arrhytmias including supraventricular arrhythmias, atrial arrhythmias, atrial flutter, atrial fibrillation; other diseases listed in Heart Disease: A Textbook of Cardiovascular Medicine, 2 Volume Set, 6th Edition, 2001, Eugene Braunwald, Douglas P. Zipes, Peter Libby, Douglas D. Zipes.

The following examples are presented to demonstrate the invention. The examples are illustrative only and are not intended to limit the scope of the invention in any way.

### EXAMPLE 1

This example shows the preparation of antibodies to the peptide SEQ ID NO: 1.

The peptide of SEQ ID NO: 1 was synthesized by the standard solid phase technique. Peptide purity was >95% by HPLC.

Conjugation of the peptide of SEQ ID NO: 1 to an immunogenic protein carrier was performed by the conventional method. The method involved the reaction of the free epsilon-amino group of the peptide SEQ ID NO: 1 to lysine group of the immunogenic carrier, in this case keyhole limpet hemocyanin (KLH). Briefly, 100 mg of the KHL was dissolved in 2 ml of water for 4 hours. The solution was dialyzed overnight against 2 liters of 0.1 M sodium phosphate pH 7.8 to remove contaminants. Aggregates were removed on spin microcentifuge. 5 mg of the peptide SEQ ID NO: 1 was added to 100 ml of the immunogenic protein carrier solution, followed by glutaraldehyde to 0.1% final, and then the pH was adjusted to 7.8 by sodium hydroxide. The mixture was incubated under gentle rotating for 12 hours at 4 degrees. The resulted mixture was stirred for 2 hours at room temperature and dialyzed twice for 4 hours against 5 liters of PBS buffer. After lyophilization, it provides the conjugate of the formula pGlu-Pro-Pro-Gly-Gly-Ser-Lys(KLH)-Val-Ile-Leu-Phe. Protein content of dialyzed conjugates and control was determined by the bicinchoninic acid assay (Pierce). Quantitation of peptide incorporation into the conjugate was determined by amino acid analysis as previously described. Shuler K R. et al., J Immunol Methods 156:137 (1992).

Mice were given subcutaneous injections of 100 μg of the conjugate and boosted 10 days later with 100 μg. Serum was collected on the 10^{th} day after the initial boost and after each subsequent boost at monthly interval. Antibodies were partially purified by 40% ammonium sulphate precipitation followed by exhaustive dialysis of the precipitate against phosphate-buffered saline (PBS). Samples of antibodies were stored at −100° C. Protein was estimated by absorbance at 280 nm.

### EXAMPLE 2

This example shows the use of antibodies to the peptide SEQ ID NO: 1 for preparation of pharmaceutical compositions.

The 50 mg of the lyophilized antibodies to the peptide of SEQ ID NO: 1 was mixed with 40 mg alpha, alpha-trehalose dihydrate, 1 mg L-histidine HCl, 0.64 mg L-histidine, 0.18 mg polysorbate 20, and 2 ml of water for injection to form a solution. The solution was lyophilized to provide preservative-free lyophilized powder for intravenous administration (Table 1).

TABLE 1
Lyophilized powder for intravenous administration

| Ingredient | Content, wt. % |
|---|---|
| Antibody to the peptide SEQ ID NO: 1 | 54 |
| α,α-Trehalose dihydrate | 39.1 |
| L-histidine HCl | 1 |
| L-histidine | 0.7 |
| polysorbate 20 | 0.2 |

To prepare a solution for intravenous administration, the liophylized powder is reconstituted in 2 ml sterile water for injections at a pH of about 6.

### EXAMPLE 3

This example shows the method for treating cancer.

Female BALB/c mice received i.p. suspension of 5×10⁵/0.05 ml of HT-29 tumor cells. The tumor bearing mice were treated i.p with the composition of example 2 in amount containing 4 mg/kg of the antibodies to the peptide SEQ ID NO: 1 or saline (control) for 7 days. The treatment significantly increased life span as compared to the control (25±7 days as compared to 18±5 days in control group (p<0.05)).

### EXAMPLE 4

This example shows the method for treating cardiovascular disease.

Female BALB/c mice were given a single intraperitoneal injection of the peptide SEQ ID NO: 1 5 μg/kg to develop myocardial hypertrophy in combination with 4 mg/kg of the antibodies to the peptide SEQ ID NO: 1 (n=10) or saline (control, n=10). The antibodies were found to completely prevent the hypertrophy of muscular and connective tissue components of the myocardium as well as myocardium wall vessels as compared to the control.

1. A pharmaceutical composition comprising an antibody that specifically binds a peptide of the formula pGlu-Pro-Pro-Gly-Gly-Ser-Lys-Val-Ile-Leu-Phe (SEQ ID NO: 1) and a pharmaceutically acceptable carrier.

2. The composition of claim 1, wherein said antibody is an isolated antibody.

3. The composition of claim 2, wherein said antibody is a monoclonal antibody.

4. A method for treating cancer in a mammal, the method comprising a step of administering to the mammal an effective amount of the pharmaceutical composition of claim 1.

5. A method for treating a cardiovascular disease, the method comprising a step of administering to the mammal an effective amount of the pharmaceutical composition of claim 1.
